(19) United States
(12) Patent Application Publication
(10) Pub. No.: US 2016/0120869 A1
(43) Pub. Date: May 5, 2016

(54) SOLID PHARMACEUTICAL DOSAGE FORM

(71) Applicant: RATIOPHARM GMBH, Ulm (DE)

(72) Inventors: Dirk LEUTNER, Munich (DE); Konstantin HOLFINGER, Munich (DE); Hans-Juergen MIKA, Bonn (DE)

(73) Assignee: ratiopharm GmbH, Ulm (DE)

(21) Appl. No.: 14/894,703

(22) PCT Filed: May 23, 2014
(86) PCT No.: PCT/EP2014/060701

§ 371 Date: Nov. 30, 2015

Related U.S. Application Data
(60) Provisional application No. 61/828,221, filed on May 29, 2013.

(30) Foreign Application Priority Data
May 29, 2013 (EP) 13169772.4

(51) Int. Cl. A61K 31/519; B65D 81/26; A61K 9/50; A61K 9/24; A61K 31/616; A61K 9/00

(52) U.S. Cl. CPC A61K 31/519; A61K 31/616; A61K 9/0053; A61K 9/50; A61K 9/209; B65D 81/264

(57) ABSTRACT

The present invention relates to a solid pharmaceutical dosage from comprising ticagrelor and ASA as pharmaceutically active agents, to a package for storing the solid pharmaceutical dosage form and to a solid pharmaceutical dosage form for the use in the treatment of certain diseases.

### PRIORITY

This application corresponds to the U.S. national phase of International Application No. PCT/EP2014/060701, filed May 23, 2014, which, in turn, claims priority to European Patent Application No. 13.169772.4 filed May 29, 2013 and U.S. Provisional Application No. 61/828,221 filed May 29, 2013, the contents of which are incorporated by reference herein in their entirety.

### FIELD OF THE INVENTION

The present invention relates to a solid pharmaceutical dosage form comprising the pharmaceutically active agents ticagrelor and ASA, to a package comprising the solid pharmaceutical dosage form and to this dosage form for the use in the treatment of certain diseases.

### BACKGROUND OF THE INVENTION

The compound [1S-[1α,2α,3β(1S*,2R*),5β]]-3-[7-[[2-(3,4-difluorophenyl)cyclopropyl]-amino]-5-(propylthio)-3H-1,2,3-triazolo[4,5-d]pyrimidin-3-yl]-5-(2-hydroxyethoxy)-cyclopentane-1,2-diol is known as ticagrelor and is represented by the formula (I) below:

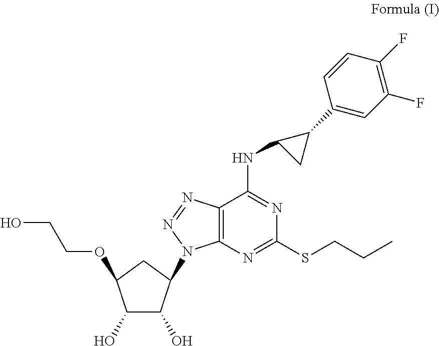

Ticagrelor can be prepared according to the methods disclosed in WO 99/05143. This document also discloses that ticagrelor exhibits activity as P2Y_{ADP} receptor antagonist.

ADP induced platelet aggregation is mediated by the P2Y_{ADP} receptor subtype located on the platelet membrane. The P2Y_{ADP}, a G-protein coupled receptor is primarily involved in mediating platelet aggregation/activation. The pharmacological characteristics of this receptor have been described by Humphries et al. in Br. J. Pharmacology (1994), 113, 1057-1063 and by Fagura et al. in Br. J. Pharmacology (1998), 124, 157-164.

Platelet adhesion and aggregation are initiating events in arterial thrombosis. Although the process of platelet adhesion to the sub-endothelial surface have an important role to play in the repair of damaged vessel walls, misdirected platelet aggregation can initiate acute thrombotic occlusion of vital vascular beds leading to events with high morbidity such as myocardial infarction and stable angina.

Ticagrelor is indicated for the prevention of thrombotic complications in patients with coronary artery, cerebrovascular or peripheral vascular disease and thrombotic events like stroke or heart attack in patients with acute coronary syndrome or myocardial infarction with or without ST elevation.

The compound 2-acetyloxybenzoic acid, is widely known as acetylsalicylic acid (ASA), was first isolated in 1897, and is now commonly used as analgesic to relieve minor aches and pains, as antipyretic to reduce fever an and as anti-inflammatory medication.

ASA irreversibly inhibits the enzyme COX, resulting in a reduced platelet production of thromboxane (TXA₂), which under normal circumstances binds platelet molecules together to create a patch over damaged walls of blood vessels. Long-term, ASA is also used at low doses, to prevent heart attacks, strokes, and blood clot formation in people at high risk of developing blood clots and has been known to prevent the progression of existing cardiovascular disease (Lewis et al. in NEJM (1983), 309 (7), 369-403).

Treatment of diseases such as acute coronary syndromes might require co-administration of antiplatelet agents having a different mode of action. A combination of ticagrelor and ASA increases efficacy and improves patient compliance with the daily dosage requirement during chronic treatment.

According to WO 01/92262 ticagrelor exists in four different crystalline forms and an amorphous form.

Pharmaceutical compositions comprising ticagrelor are disclosed in WO 2008/024044 and WO 2008/024045. It is disclosed that these compositions, which may contain up to 50% by weight of the active ingredient, are suitable for oral administration and that they release substantially all of the active ingredient. These preparations are produced according to a conventional manner using a wet granulation process.

WO 2011/076749 provides a solid dosage form comprising ticagrelor. This solid dosage form may comprise an additional anti-thrombotic compound including ASA. However, it has now surprisingly been found by the present invention, that the dosage forms disclosed in WO 2011/076749 do not show any long term stability. Both pharmaceutically active agents ticagrelor as well as ASA were degraded and decomposed extensively within the solid pharmaceutical dosage form after several weeks of storage, particularly, when stored under ICH conditions. Thus, the solid pharmaceutical dosage forms provided in the prior art are not suitable as solid pharmaceutical dosage forms placed on the market.

WO 2012/164286 relates to ticagrelor/ASA co-crystals and the preparation thereof. However, the application provides no data regarding the stability of the ticagrelor/ASA co-crystals or the co-crystals incorporated in solid pharmaceutical dosage forms. Furthermore, it is not clear whether co-crystals are supposed to be more stable than physical mixtures of both drugs. For the process of manufacture a solid pharmaceutical dosage form an additional time and resources consuming step is needed comprising the preparation of the co-crystals, which can be avoided by providing a solid pharmaceutical dosage form using the readily available pharmaceutical agents.

CN 102228691 discloses aspirin and anticoagulant containing pharmaceutical oral compositions, whereas the anticoagulant substance comprises ticagrelor. Although this composition can allegedly also be used for treating or preventing cardiovascular and cerebrovascular diseases the document does not describe a specific handling of the two active ingredients but rather mixes them together, similar to the teachings of the earlier WO 2011/076749.

In the formulation of solid pharmaceutical dosage forms, it is important that the pharmaceutically active agent is in a form, in which it can be conveniently handled and processed. This is of importance, not only from the point of view of obtaining a commercially viable manufacturing process, but also from the point of subsequent manufacture of pharmaceutical formulations comprising the pharmaceutically active agent. Chemical stability, solid state stability, and shelf life of the pharmaceutically active agents are also very important factors. The pharmaceutically active agent, and compositions containing it, should be capable of being effectively stored over appreciable periods of time, without exhibiting a significant change in the active component's physicochemical characteristics, for example its chemical composition, density, hygroscopicity and solubility.

### SUMMARY OF THE INVENTION

Dosage forms comprising ASA and ticagrelor are known. It has surprisingly been found, that the dosage forms disclosed in the patent application WO 2011/076749 do not show any long term stability of the combined pharmaceutical active agents. It was, however, not evident whether the manufacturing process, the excipients, or any other factors influence the stability of the combined pharmaceutically active agents. It has now surprisingly been found, that the long term stability of the pharmaceutical active agents can be increased significantly, when the pharmaceutical agents are present in different compartments within the solid pharmaceutical dosage form.

While applicants do not wish to be bound to any theory it is speculated that both pharmaceutically active agents may possibly cause mutual degradation when they are in direct contact with each other.

The dissolution rate of pharmaceutically active agents is an essential condition for their safety and bioavailability. It would be desirable to provide a solid pharmaceutical dosage form which not only stabilizes the pharmaceutically active agents within the solid pharmaceutical dosage form but additionally provides separate dissolution rates of both pharmaceutically active agents, ticagrelor and ASA. Moreover, it would be desirable to increase the stability of the solid pharmaceutical dosage form under forced degradation conditions. These and other problems are solved by the present invention.

Thus, the present invention relates to a solid pharmaceutical dosage form comprising the pharmaceutically active agents
a) ticagrelor or a pharmaceutically acceptable salt, solvate or ester thereof and
b) acetylsalicylic acid or a pharmaceutically acceptable salt, solvate or ester thereof,
   characterized in that said active agents are present in different compartments within the solid pharmaceutical dosage form.

### BRIEF DESCRIPTION OF THE FIGURES:

FIG. 1 is a line graph depicting the dissolution rate of the tablet based on Example 1.

FIG. 2 is a line graph depicting the dissolution rate of the tablet based on Example 2.

FIG. 3 is a line graph depicting the dissolution rate of the tablet based on Example 5.

FIG. 4 is a bar graph depicting the results of stability testing.

### DETAILED DESCRIPTION OF THE PREFERRED EMBODIMENTS:

When in the following it is referred to ticagrelor or ASA, it should be understood that such reference relates to the free active agent or a pharmaceutically acceptable salt, solvate or ester thereof.

The term “compartments” as used herein refers to separate units or domains within the solid pharmaceutical dosage form, such as layers or particles. Each compartment comprises none or one of said two pharmaceutically active agents and, if it contains one of said two active agents, it is free of the other of said active agents. A compartment either contains ticagrelor as pharmaceutically active agent, ASA as pharmaceutically active agent or is free of these two pharmaceutically active agents. Each compartment can, however, contain further active agent(s) other than ticagrelor and ASA as will be explained below.

The compartments are designed in a way that they reduce or inhibit interaction of the two active agents ASA and ticagrelor, in particular that they reduce or inhibit contact between said two active agents.

A compartment may contain only active agent, active agent with excipient(s) or may be free of active agent and therefore may be composed of excipient(s) only.

In the present application by “layers” it should be understood that, according to one embodiment of the present invention, the solid pharmaceutical dosage form is made up of distinct layers, and furthermore the compartments comprising either the pharmaceutically active agents ticagrelor or ASA are separated into distinct layers.

In one embodiment of the present invention the active agents ticagrelor and ASA are present in two different layers being adjacent to each other.

In another embodiment of the present invention the layers containing the active agents ticagrelor and ASA at least partially are separated from each other by at least one inert layer which is free of the active agents ticagrelor and ASA but which may contain at least one other pharmaceutically active ingredient. Thus, “inert” only refers to the feature that the layer is free of the active agents ticagrelor and ASA.

The inert layer preferably is shaped such that it completely separates adjacent compartments from each other.

In the present application by “particles” it should be understood that, according to one embodiment of the present invention, the solid pharmaceutical dosage form is made up of distinct particles, and furthermore the compartments comprising either the pharmaceutically active agents ticagrelor or ASA are separated into distinct particles, wherein the ticagrelor containing particles and/or the ASA containing particles are coated.

Preferably, both the ticagrelor containing particles and the ASA containing particles are coated. More preferably, the coating is at least one inert coating which is free of the active agents ticagrelor and ASA but which may contain at least one other pharmaceutically active ingredient. As particles active ingredient particles or granules, pellets or microtablets containing the active agent are for example suitable. The particles, optionally together with inert particles (i.e. particles which do not contain any ticagrelor and ASA but optionally any other pharmaceutically active ingredient), may for example be filled into capsules or sachets or may be compressed into tablets.

The thickness of the inert layer or the coating is adjusted to improve the stability of the active agents present in the adjacent layers or particles respectively. A person skilled in the art can readily determine the stabilizing effect of the inert layer or coating based on the analysis of a stability test relating to appropriate solid pharmaceutical dosage forms comprising ticagrelor and ASA.

Any compound or mixture of compounds suitable for separating two compartments within a solid pharmaceutical dosage form can be used for preparing the inert layer or coating. The inert layer or coating can be prepared from the same or different compounds. The inert layer can for example be prepared from compounds, such as fillers and binders which are usually used in the preparation of pharmaceutical dosage forms. For the coating film forming compounds as known to the person skilled in the art are for example suitable. The layers, particles and coatings in the pharmaceutical dosage form of the invention can comprise the same or different ingredients except for the active agents ticagrelor and ASA.

Another advantage of the present invention is that the ingredients of the layers, particles and coatings can be selected such as to tailor the release profile of the pharmaceutical dosage form. For example, the ingredients can be selected in order to prepare a dosage form having an immediate-release profile or a modified-release profile, such as an extended-release profile, as explained in more detail below. A further advantage is that for the two active agents ticagrelor and ASA different release profiles can be selected, if desired.

For example, the compartments comprising the active agents may contain these active agents within a matrix forming excipient, thus forming for example an immediate-release matrix or an extended-release matrix. If the compartments are in the form of particles, the release profile may be adjusted by the coating of the particles. Furthermore, the release profile may be adjusted by an additional coating which may surround the pharmaceutical dosage form which can, for example, be present in the form of a coated tablet.

Suitable matrix forming excipients and film forming excipients are known to the person skilled in the art. Suitable matrix forming excipients are for example polyacrylic acid (Carbomer), polyvinylacetate, polyvinylpyrrolidone, polyethylene oxide, polyethylene glycol, cellulose derivates, such as hypromellose (HPMC), methyl cellulose, hydroxypropyl cellulose, hydroxyethyl cellulose, methylhydroxyethyl cellulose, ethylhydroxyethyl cellulose, methylhydroxypropyl cellulose, carboxymethylcellulose sodium, cellulose acetate succinate, cellulose acetate butyrate, cellulose acetate phthalate, cellulose acetate trimellitate, hydroxypropylmethyl cellulose acetate succinate, hydroxypropylmethyl cellulose phthalate, ethyl cellulose, sodiumethyl cellulose sulfate, carboxymethylethyl cellulose sodium, cellulose acetate, polyvinyl acetate phthalate, methacrylic acid (acrylate-co)polymers, such as Eudragit® L or S, Eudragit® RL or RS and Eudragit® NE or NM, starch derivatives, such as hydroxypropyl starch, fats, waxes, oils, fatty acids, fatty alcohols or glycerol derivatives, such as glycerol dibehenate, glycerol distearate and hydrogenated vegetable oil, carrageenan, chitosan, tannin, sodium alginate, alginic acid and acacia.

Suitable excipients for forming the inert layer are for example fillers, such as lactose, saccharose, glucose, mannitol, sorbitol, starches, modified starches, cellulose, calcium hydrogenphosphate, calcium carbonate and calcium sulfate. These fillers may also be used as matrixforming excipients in the active agent containing layers or particles. Furthermore, the above exemplified matrix forming excipients are also suitable for forming the inert layer.

Suitable film forming excipients for immediate-release preparations are for example cellulose derivates such as hypromellose (HPMC), methyl cellulose, hydroxypropyl cellulose, hydroxyethyl cellulose, methyl hydroxyethyl cellulose, ethylhydroxyethyl cellulose, methylhydroxypropyl cellulose and carboxymethyl cellulose sodium, polyvinylpyrrolidone, polyvinylacetate, sugars such as saccharose, glucose, sorbit and lactose, methacrylic acid (acrylate-co)polymers, such as Eudragit® E and polyvinyl alcohol polyethylen glycol copolymer (Kollicoat IR).

Suitable film forming excipients for modified-release preparations are for example methacrylic acid (acrylate-co)polymers, such as Eudragit® L or S, Eudragit® RL or RS and Eudragit® NE or NM, cellulose derivates, such as cellulose acetate succinate, cellulose acetate butyrate, cellulose acetate phthalate, cellulose acetate trimellitate, hydroxypropylmethyl cellulose acetate succinate, hydroxypropylmethyl cellulose phthalate, ethyl cellulose, sodium ethyl cellulose sulfate, carboxymethylethyl cellulose sodium and cellulose acetate, hydroxypropyl starch, schellack, polyvinylacetate, polyvinylacetate phtalate, polyacrylic acid (Carbomer), fats, waxes, oils, fatty alcohols, fatty acids and glycerol derivatives, such as exemplified above.

The pharmaceutical dosage form according to the present invention can further comprise in any of its layers or particles additional excipients and adjuvants, which are pharmaceutically acceptable. In addition general coating materials might further be comprised in the pharmaceutical dosage, which are preferably applied as a coating to the pharmaceutical dosage form of the present invention. Such further excipients and adjuvants are known to the person skilled in the art. In this regard it can be referred to the standard textbook by Fiedler (“Lexikon der Hilfsstoffe für Pharmazie, Kosmetik and angrenzende Gebiete”, 5^{th} ed., 2002) and to the “Handbook of Excipients”, edited by the American Pharmaceutical Association and Dr. Arthur H. Kibbe, 3^{rd} ed., 2000.

In a preferred embodiment, each compartment of the solid pharmaceutical dosage form containing an active agent additionally contains at least one excipient wherein the ratio between active agent and excipient(s) is 1 to at least 0.3, preferably 1 to at least 0.8, more preferably 1 to at least 1, such as in the range of 1:0.3 to 1:20, preferably in the range of 1:1 to 1:10.

In another preferred embodiment each layer or particle comprises at least more than 5% by weight, more preferably more than 10% by weight, even more preferably more than 15% by weight, most preferably more than 20% by weight of either the active agent ticagrelor or ASA in its free base or acid form, each based on the total weight of the layer or particles (including its coating).

In particular the pharmaceutical dosage form according to the present invention comprises one or more excipients in the form of diluents, binders, disintegrants, glidants, lubricants and/or coating materials and optionally colorants and/or surfactants.

As diluent one or more components can be used, which contribute part of the dosage form to reach the necessary total mass of the dosage form. Preferable diluents are inorganic phosphates, like dibasic calcium phosphate, or sugars or sugar analogues and derivatives thereof, in particular lactose, such as lactose monohydrate including but not limited to the commercially available tablettose or water-free lactose, dextrose, saccharose, maltodextrin, or sugar alcohols including but not limited to sorbitol, mannitol, isomalt (E953). Cellulose like microcrystalline cellulose or powdered celluloses are also preferable diluents according to the present invention.

As disintegrant one or more components can be used, which decompose the tablet in the gastrointestinal fluid. Preferable disintegrants are microcrystalline cellulose, alginates, pregelatinized starch, modified starch like croscarmellose, sodium carboxymethyl starch and crosslinked PVP and crospovidon.

Additional excipients to be used in the solid pharmaceutical dosage form are binders, which are compounds able to bind together active ingredient and excipients in a mixture. As binder to be used in the pharmaceutical dosage form of the present invention polyvinylpyrrolidon (PVP) preferably having the molecular weight of 10,000 to 60,000 g/mol and copolymers of PVP preferably having a molecular weight of 40,000 to 70,000 g/mol, microcrystalline cellulose and hydroxypropylmethyl cellulose are especially preferred.

Additional excipients to be used in the pharmaceutical dosage form of the present invention are glidants, which are added to a powder to improve its flowability, such as silicium dioxide or fumed silicium dioxide, and/or amphipatic wetting agents, such as sodium dodecylsulfate (SDS), glycerol monostearate, triglycerides or glycerol behenate.

Optionally, as lubricants can be used in the pharmaceutical dosage forms of the present invention fatty acids or fatty acid derivates, such as alkali and earth alkali salts of stearic, lauric and/or palmitic acid. Sodium stearyl fumarate and magnesium stearate are preferred.

Ticagrelor and/or ASA can be amorphous or in any other polymorphic crystalline form as disclosed for example in WO 01/92262. The polymorphic forms include hydrates, solvates, etc. Moreover, ticagrelor and/or ASA can be present in its free base form or in the form of any pharmaceutically acceptable salt or ester known to a person skilled in the art. As salts those of inorganic or organic acids can be exemplified, such as hydrochloride, sulfate, mesylate, tosylate, besylate, etc. As esters those of organic acids or alcohol can be exemplified, such as acetate or ethanol. Independently of the polymorphic form ticagrelor can be present in micronized form.

In an embodiment of the present invention, the solid pharmaceutical oral dosage form comprises ticagrelor or a pharmaceutically acceptable salt, solvate or ester thereof, in an amount therapeutically equivalent to ticagrelor free base, an amount from 10 to 400 mg, preferably an amount from 50 to 200 mg, particularly an amount of 90 mg or 180 mg.

In a further embodiment of the present invention the solid pharmaceutical dosage form comprises ASA or a pharmaceutically acceptable salt, solvate or ester thereof, in an amount therapeutically equivalent to ASA free acid in an amount from 5 to 500 mg, preferably in an amount from 10 to 375 mg, more preferably in an amount from 20 to 100 mg, particularly in an amount from 37.5 mg to 100 mg.

A particular advantage of the solid pharmaceutical dosage form of this invention is its advantageous dissolution profile. An essential condition for the resorption of the pharmaceutically active agents and thus of their bioavailability depends on the dissolution of the pharmaceutically active agent from the solid pharmaceutical dosage form. There are different dissolution profiles for solid pharmaceutical dosage forms. Generally, controlled-release systems (modified-release systems) can be categorized into two groups based on their actions. After ingestion, extended-release formulations (ER) dissolve the total dose over an extended time frame. Delayed-release systems provide steady dosing after passage through the stomach. Controlled-drug delivery systems aim to maintain plasma concentration of drugs within the therapeutic window for a longer period of time, thereby to ensure sustained therapeutic action. “Modified-release” means that the release of the drug from the dosage form has been modified in some way with respect to an immediate-release (IR) delivery of the same drug.

In one embodiment of the present invention, the pharmaceutically active agents ticagrelor and ASA are released from the solid pharmaceutical dosage form with respect to an IR delivery. In a further embodiment of the present invention, the pharmaceutically active agent ticagrelor is released from the solid pharmaceutical dosage form with respect to an ER delivery and ASA with respect to an IR delivery.

In a first embodiment of the present invention, the solid pharmaceutical dosage form dissolves more than 60%, preferably more than 75%, most preferably more than 80% of ticagrelor or a pharmaceutically acceptable salt, solvate or ester thereof, and/or dissolves more than 80%, preferably more than 90%, most preferably more than 95% of ASA or a pharmaceutically acceptable salt, solvate or ester thereof from the dosage form not later than 60 minutes from the start of the test using an USP apparatus I (Basket System) using 900 ml water containing 0.3% SDS at 37° C. as dissolution medium and a rotation speed of 75 rpm.

In a second embodiment of the present invention, the solid pharmaceutical dosage form dissolves more than 50%, preferably more than 60% of ticagrelor or a pharmaceutically acceptable salt, solvate or ester thereof, and/or dissolves more than 80%, preferably more than 90% of ASA or a pharmaceutically acceptable salt, solvate or ester thereof from the dosage form not later than 30 minutes from the start of the test using an USP apparatus I (Basket System) using 900 ml water containing 0.3% SDS at 37° C. as dissolution medium and a rotation speed of 75 rpm.

In a third embodiment of the present invention, the solid pharmaceutical dosage form dissolves more than 30%, preferably more than 40% of ticagrelor or a pharmaceutically acceptable salt, solvate or ester thereof, and/or dissolves more than 40%, preferably more than 50% of ASA or a pharmaceutically acceptable salt, solvate or ester thereof from the dosage form not later than 15 minutes from the start of the test using an USP apparatus I (Basket System) using 900 ml water containing 0.3% SDS at 37° C. as dissolution medium and a rotation speed of 75 rpm.

The above first and second, first and third, second and third, or first, second and third embodiment may be combined to further preferred embodiments.

In a fourth embodiment of the present invention, the solid pharmaceutical dosage form dissolves at the most 90%, preferably at the most 80%, most preferably at the most 75% of ticagrelor or a pharmaceutically acceptable salt, solvate or ester thereof, and/or dissolves at the most 95%, preferably at the most 90%, most preferably at the most 87% of ASA or a pharmaceutically acceptable salt, solvate or ester thereof from the dosage form within 10 hours from the start of the test as determined by USP apparatus I (Basket System) using 900 ml water containing 0.3% SDS at 37° C. as dissolution medium and a rotation speed of 75 rpm.

In a fifth embodiment of the present invention, the solid pharmaceutical dosage form dissolves at the most 50%, preferably at the most 40%, of ticagrelor or a pharmaceutically acceptable salt, solvate or ester thereof, and/or dissolves at the most 60%, preferably at the most 50%, of ASA or a pharmaceutically acceptable salt, solvate or ester thereof from the dosage form within 4 hours from the start of the test as determined by USP apparatus I (Basket System) using 900 ml water containing 0.3% SDS at 37° C. as dissolution medium and a rotation speed of 75 rpm.

In a sixth embodiment of the present invention, the solid pharmaceutical dosage form dissolves at the most 40%, preferably at the most 30%, of ticagrelor or a pharmaceutically acceptable salt, solvate or ester thereof, and/or dissolves at the most 45%, preferably at the most 30%, of ASA or a pharmaceutically acceptable salt, solvate or ester thereof from the dosage form within 2 hours from the start of the test as determined by USP apparatus I (Basket System) using 900 ml water containing 0.3% SDS at 37° C. as dissolution medium and a rotation speed of 75 rpm.

The above fourth and fifth, fourth and sixth, fifth and sixth, or fourth, fifth and sixth embodiments may be combined to further preferred embodiments.

In a seventh embodiment of the present invention, the solid pharmaceutical dosage form dissolves at the most 95%, preferably at the most 90%, most preferably at the most 85% of ticagrelor or a pharmaceutically acceptable salt, solvate or ester thereof from the dosage form within 20 hours, and/or dissolves more than 60%, preferably more than 70%, most preferably more than 80% of ASA or a pharmaceutically acceptable salt, solvate or ester thereof from the dosage form not later than 60 minutes, both from the start of the test using an USP apparatus I (Basket System) using 900 ml water containing 0.3% SDS at 37° C. as dissolution medium and a rotation speed of 75 rpm.

In an eighth embodiment of the present invention, the solid pharmaceutical dosage form dissolves at the most 60%, preferably at the most 50%, most preferably at the most 45% of ticagrelor or a pharmaceutically acceptable salt, solvate or ester thereof from the dosage form within 10 hours, and/or dissolves more than 60%, preferably more than 70%, most preferably more than 80% of ASA or a pharmaceutically acceptable salt, solvate or ester thereof from the dosage form not later than 60 minutes, both from the start of the test using an USP apparatus I (Basket System) using 900 ml water containing 0.3% SDS at 37° C. as dissolution medium and a rotation speed of 75 rpm.

In a ninth embodiment of the present invention, the solid pharmaceutical dosage form dissolves at the most 35%, preferably at the most 30%, most preferably at the most 20% of ticagrelor or a pharmaceutically acceptable salt, solvate or ester thereof from the dosage form within 4 hours, and/or dissolves more than 60%, preferably more than 70%, most preferably more than 80% of ASA or a pharmaceutically acceptable salt, solvate or ester thereof from the dosage form not later than 60 minutes, both from the start of the test using an USP apparatus I (Basket System) using 900 ml water containing 0.3% SDS at 37° C. as dissolution medium and a rotation speed of 75 rpm.

The above seventh and eighth, seventh and ninth, eighth and ninth, or seventh, eighth and ninth embodiments may be combined to further preferred embodiments.

The solid pharmaceutical dosage form of the present invention preferably is an oral pharmaceutical dosage form, such as a tablet, capsule, granules, pellets or sachets. The pharmaceutical dosage form can be prepared by methods known in the art, such as tabletting by granulation or direct compression.

The compression of the compartments to tablets can be carried out using a conventional tabletting machine or a rotary compression machine. The tablets may vary in shape and can be, for example, round, oval, oblong, cylindrical or any other suitable shape. The layers may also vary in size depending on the amount and concentration of the therapeutic agents. The tablet of the invention is preferably in the form of a sandwich structure which includes one layer containing the active agent ASA, another layer containing the active agent ticagrelor, and one or more intermediate or middle barrier layers or other functional layers which separate the ASA and ticagrelor layers and minimizes, preferably inhibits, interaction between the ingredients in these layers.

Optionally, the tablet may include an ASA layer and a ticagrelor layer separated by an intermediate or middle barrier layer with an additional layer or layers applied to the outer face of the ticagrelor layer, the ASA layer or both. These additional layers can be for aesthetic purposes, to disguise the taste of the drug substances, and/or to provide for delivery of additional active materials, such as buffers or other ingredients.

The granules resulting from the present invention might be compressed into a tablet, filled in to hard capsules, sachets, stick packs, or processed into Multi Unit Pharmaceutical Systems (MUPS).

The pharmaceutical dosage form according to the present invention comprising ticagrelor and ASA may be present in form of a tablet which is coated with one or more coating materials. The coating materials are not particularly limited and are known to the person skilled in the art. As far as it is herein referred to a dissolution profile, the dissolution profile is that of an uncoated tablet, if the tablet is not coated, and that of a coated tablet, if the tablet is coated.

In an alternative embodiment of the present invention, the solid pharmaceutical dosage form contains ticagrelor/ASA in combination with a further anti-thrombotic agent and a process of forming the same. The anti-thrombotic compound is selected from anti-platelet agents, anticoagulant agents and fibrinolytic agents. Anti-thrombotic agent selected from antiplatelet agents include clopidogrel, ticlopidine, dipyridamole, GPIIb/IIIa antagonists; anti-coagulants such as thrombin inhibitors, warfarin, factor Xa inhibitors, heparin; and fibrinolytic agents including but not limited to, streptokinase and tenecteplase. The combination partner might be present in the same compartment as either one or both of the active agents ASA or ticagrelor, in the separating layer comprising neither of the active agents ASA and ticagrelor, and/ or in the coating.

Respective formulations comprising ticagrelor/ASA and/or other antithrombotic agent may be administered, sequentially, separately and/or simultaneously, over the course of treating the relevant condition, which condition may be acute or chronic.

A further aspect of the present invention is that the solid pharmaceutical dosage form can be additionally stabilized when stored in a package.

A particular advantage of the present invention is a package, which increases the stability of the solid pharmaceutical dosage form as such and under forced degradation conditions. Forced degradation conditions are standardized by the International Conference on Harmonisation of Technical Requirements for Registration of Pharmaceuticals for Human Use (ICH).

In one embodiment the present invention relates to a package comprising the solid pharmaceutical dosage, wherein the packaging material and/ or the desiccant stabilizes the solid pharmaceutical dosage form, preferably when stored at a temperature at 40° C. and by 75% RH (relative humidity), or at a temperature at 25° C. and by 60% RH. The package is preferably made of water-impermeable material, like aluminum or polyethylene. Aluminum for example provides a good barrier to moisture, vapor and gases. The term “package” includes all different packs suitable to pack the solid pharmaceutical dosage forms, preferable packs are aluminum blister packs, aluminum pack, bottles and small cylindrical containers. Preferably, the water-impermeable pack comprises a desiccant, wherein the desiccant can be selected from the group consisting of silica gel, betonite clay, molecular sieve and activated carbon, preferably silica gel and molecular sieve, most preferably molecular sieve.

In one embodiment of the present invention, the package is a water-impermeable pack, preferably an aluminum pack, an aluminum blister pack or polyethylene pack.

In a further embodiment the present invention relates to a water-impermeable pack comprising a desiccant.

In a further embodiment the solid oral dosage form is used in the treatment of coronary, cerebrovascular or peripheral diseases and thrombotic events such as stroke or heart attack preferably in patients with acute coronary syndrome or myocardinal infarction.

The invention will now be further explained by way of examples which are not construed to be limited to any extend or interpretation.

### EXAMPLE 1

### Comparative Example (Direct Compression)

| | | amount | |
|---|---|---|---|
| | ingredient | [mg] | percentage |
| 1 | Ticagrelor (adjusted to potency of 98.53%) | 182.69 | 28.01 |
| 2 | ASA | 75.00 | 11.50 |
| 3 | Silica, fumed (AEROSIL 200) | 7.50 | 1.15 |
| 4 | RetaLac (Lactose monohydrate co-processed | 360.0 | 55.20 |
| | with hypromellose 1:1) | | |
| 5 | Silica, fumed (AEROSIL 200) | 9.00 | 1.38 |
| 6 | Magnesium stearate | 18.0 | 2.76 |
| | total | 652.19 | 100.0 |

ASA is ground with 10 mass per cent silica (3). Ticagrelor is mixed with RetaLac, Silica (5) and magnesium stearate. Both powder blends are mixed together for 5 minutes and compressed to biconvex tablets and stored in aluminium/aluminium blister packs at ICH conditions.

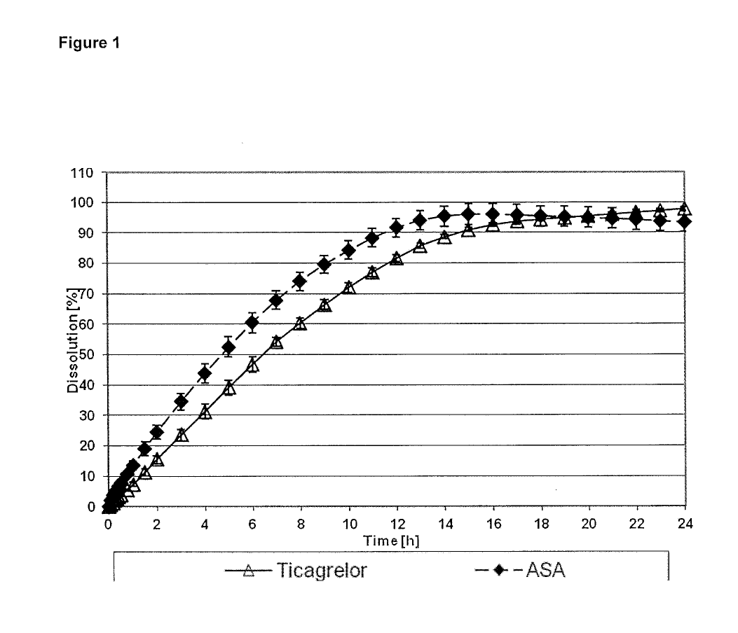

A dissolution test has been carried out with an USP apparatus I (Basket System) using 900 ml water containing 0.3% sodium dodecylsulfate at 37° C. as dissolution medium and a rotation speed of 75 rpm. The pharmaceutically active agents ticagrelor and ASA were released from the solid pharmaceutical dosage form with respect to an ER delivery. The result is shown in FIG. 1. The dissolution of ticagrelor is expressed by triangles and that of ASA by squares.

### EXAMPLE 2

### Coated Granules

| | | amount | |
|---|---|---|---|
| | ingredient | [mg] | percentage |
| 1 | Ticagrelor (adjusted to potency of 98.53%) | 91.34 | 27.05 |
| 2 | ASA | 37.5 | 11.10 |
| 3 | HPMC ready mix (Sepifilm LP010: HPMC | 1.88 | 0.56 |
| | with MCC and stearic acid) | | |
| 4 | Water, purified | q.s. | q.s. |
| 5 | Hard fat (Massa estarinum 299) | — | — |
| 6 | Lactose monohydrate (Tablettose 80) | 120.0 | 35.53 |
| 7 | Microcrystalline cellulose (Avicel PH-101) | 55.0 | 16.29 |
| 8 | Sodium carboxymethyl starch (Ac-Di-Sol) | 24.0 | 7.11 |
| 9 | Magnesium stearate | 8.00 | 2.37 |
| | total | 337.72 | 100.0 |

Manufacture:

ASA is coated with a 10% solution of Sepifilm LP010 in water. The granules are dried for 24h at 40° C. in a cabinet dryer and sieved afterwards (mesh size 500 μm). Ticagrelor and all other excipients were ground. After a dry compaction step and blending the final powder mixture is compressed to biconvex tablets and stored in aluminium/aluminium blister packs.

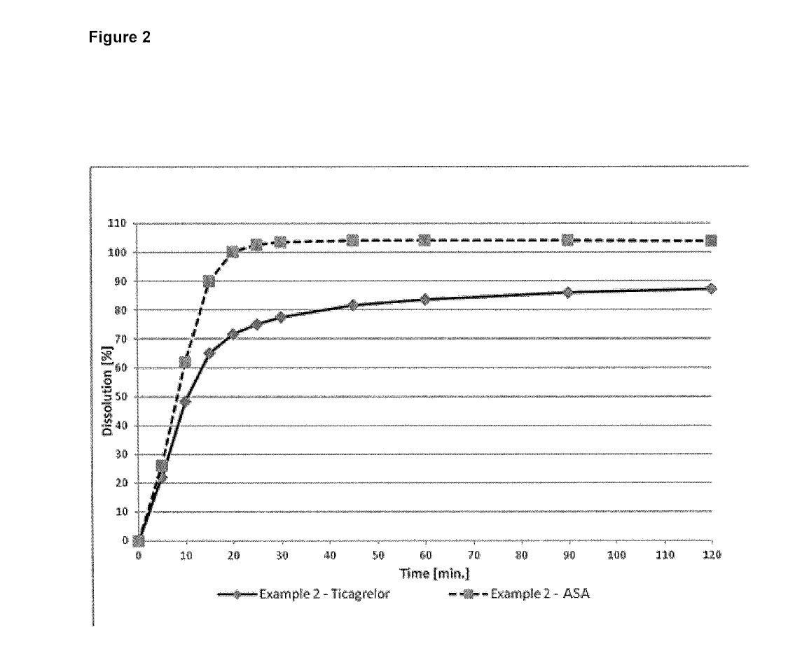

The tablet prepared according to example 2 has the dissolution rate as shown in FIG. 2. The dissolution test has been carried out as described in example 1.

The pharmaceutically active agents ticagrelor and ASA are released from the solid pharmaceutical dosage form with respect to an IR delivery.

### EXAMPLE 3

### Bilayer Tablet

| | ingredient | amount [mg] | percentage |
|---|---|---|---|
| 1.1 | Ticagrelor (adjusted to potency of | 182.69 | 20.53 |
| | 98.53%) | | |
| 1.2 | Lactose monohydrate (Tablettose 80) | 120.00 | 26.98 |
| 1.3 | MCC (Avicel PH-101) | 55.00 | 12.36 |
| 1.4 | Sodium carboxymethyl starch (Ac-Di-Sol) | 24.00 | 5.40 |
| 1.5 | Stearic acid | 8.00 | 1.80 |
| 2.1 | ASA | 37.50 | 8.43 |
| 2.2 | Corn starch, pregelatinized (Starch 1500) | 1.35 | 1.35 |
| 2.3 | MCC (Acivel PH-101) | 103.00 | 23.15 |
| | total | 444.84 | 100.0 |

Manufacturing:

Layer 1: Ticagrelor is ground with excipients 1.2-1.4 and mixed. Stearic acid is added and mixed for another 3 minutes. Layer 2: ASA is ground with excipients 2.2 and 2.3 and mixed. Bilayer tablets are pressed with both powder blends and stored in alu/alu blister packs.

### EXAMPLE 4

### Trilayer Tablet, Immediate Release

| | | amount | |
|---|---|---|---|
| | ingredient | [mg] | percentage |
| 1.1 | Ticagrelor (adjusted to potency of 98.53%) | 91.34 | 17.74 |
| 1.2 | Microcrystalline cellulose (Avicel PH-112) | 55.0 | 10.68 |
| 1.3 | Mannitol (Pearlitol 200SD) | 120.0 | 23.31 |
| 1.4 | Sodium carboxymethyl starch (Ac-Di-Sol) | 24.0 | 4.66 |
| 1.5 | Stearic acid | 8.0 | 1.55 |
| 2 | Vegetable oil, hydrogenated (Lubritab) | 70.0 | 13.60 |
| 3.1 | ASA | 37.5 | 7.28 |
| 3.2 | Corn starch, pregelatinized (PC-10) | 6.0 | 1.17 |
| 3.3 | Microcrystalline cellulose (Avicel PH-112) | 103.00 | 20.01 |
| | total | 514.84 | 100.0 |

Manufacturing:

Layer 1: Ticagrelor is mixed with excipients 1.2-1.4 for 10 minutes. Stearic acid is added and mixed for another 3 minutes. Layer 2: Lubritab is sieved (mesh size 500 pm). Layer 3: ASA is mixed with excipients 3.2 and 3.3. Trilayer tablets were pressed and stored in HDPE bottles with desiccant.

### EXAMPLE 5

### Trilayer Tablet, Modified Release

| | | amount | |
|---|---|---|---|
| | ingredient | [mg] | percentage |
| 1.1 | Ticagrelor (adjusted to potency of 98.53%) | 182.69 | 22.18 |
| 1.2 | HPMC (Methocel K4M CR) | 180.0 | 21.85 |
| 1.3 | Mannitol (Pearlitol 200SD) | 180.0 | 21.85 |
| 1.4 | Silica, fumed (AEROSIL 200) | 5.5 | 0.67 |
| 1.5 | Sodium stearyl fumarate (Pruv) | 5.5 | 0.67 |
| 2 | Vegetable oil, hydrogenated (Lubritab) | 120.0 | 14.57 |
| 3.1 | ASA | 75.0 | 9.11 |
| 3.2 | Corn starch, pregelatinized (PC-10) | 6.0 | 0.73 |
| 3.3 | Microcrystalline cellulose (Avicel PH-112) | 67.5 | 8.19 |
| 3.4 | Sodium stearyl fumarate (Pruv) | 1.5 | 0.18 |
| | total | 823.69 | 100.0 |

Manufacturing:

Layer 1: Ticagrelor is mixed with excipients 1.2-1.4 for 10 minutes. Sodium stearyl fumarate is added and mixed for another 3 minutes. Layer 2: Lubritab is sieved (mesh size 500 pm). Layer 3: ASA is mixed with excipients 3.2 and 3.3 for 10 minutes. Pruv is added and mixed for another 3 minutes. Trilayer tablets were pressed and stored in HDPE bottles with desiccant.

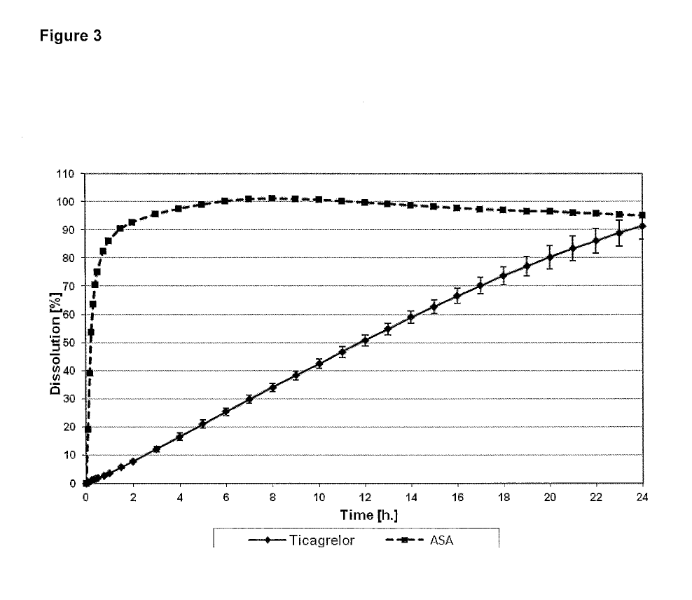

The tablet prepared according to example 5 has the dissolution rate as shown in FIG. 3. The dissolution test has been carried out as described in example 1. The pharmaceutically active agent ticagrelor is released from the solid pharmaceutical dosage form with respect to an extended-release delivery and ASA with respect to an IR delivery.

### EXAMPLE 6

### Trilayer Tablet

Example 6 is a trilayer tablet using ASA crystals coated with Eudragit® L 30D-55. It is produced in analogy to example 4. Except of 37.5 mg ASA 38.46 mg coated ASA equivalent to 37.5 mg ASA was used. This totals to a tablet weight of 515.8 mg.

### EXAMPLE 7

### Results of Stability

The stability tests were carried out under forced degradation conditions as follows:

| | 4 weeks 40° C./ | 4 weeks 25° C./ |
|---|---|---|
| Increase in RS by HPLC, area-% | 75% RH | 60% RH |
| Example 1 (comparative) | 6.9%/41.0% | 0.7%/5.9% |
| Increase in total impurities | | |
| Ticagrelor/ASA | | |
| Alu/alu blister pack | | |
| Example 2 | 9.6%/27.7% | 1.5%/4.0% |
| Increase in total impurities | | |
| Ticagrelor/ASA | | |
| Alu/alu blister pack | | |

| | 4 weeks | 12 weeks | 12 weeks |
|---|---|---|---|
| Increase in RS by HPLC, | 40° C./ | 40° C./ | 25° C./ |
| area-% | 75% RH | 75% RH | 60% RH |
| Example 3 | 0.3%/0.7% | 1.0%/1.4% | Not analyzed |
| Increase in total impurities | | | |
| Ticagrelor/ASA | | | |
| Alu/alu blister pack | | | |
| Example 4 | 0.0%/0.0% | 0.1%/0.0% | Not analyzed |
| Increase in total impurities | | | |
| Ticagrelor/ASA | | | |
| HDPE bottle with desiccant | | | |
| Example 5 | 0.0%/0.4% | 0.1%/0.3% | 0.0%/0.1% |
| Increase in total impurities | | | |
| Ticagrelor/ASA | | | |
| HDPE bottle with desiccant | | | |

| | 2 weeks 40° C./ | 4 weeks 40° C./ |
|---|---|---|
| Increase in RS by HPLC, area-% | 75% rH | 75% rH |
| Example 6 | 0.0%/0.7% | 0.0%/1.0% |
| Alu/alu blister pack | | |
| Increase in total impurities | | |
| Ticagrelor/ASA | | |
| Example 6 | 0.0%/0.2% | 0.0%/0.6% |
| HDPE bottle with desiccant | | |
| Increase in total impurities | | |
| Ticagrelor/ASA | | |

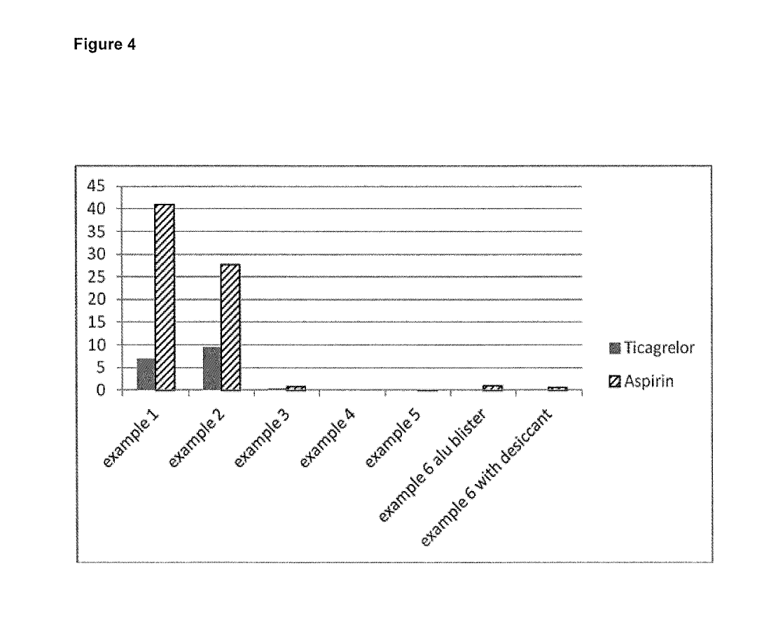

FIG. 4 provides an overview of the quantitative degradation of the pharmaceutical active agents ticagrelor and ASA formulated in a solid pharmaceutical dosage form according to examples 1-5, when stored at 40° C. and 75% RH for four weeks. The grey column expresses the ticagrelor degradation, the dashed the ASA degradation.

1. A solid pharmaceutical dosage form comprising different first and second compartments, wherein said first compartment contains the pharmaceutically active agent ticagrelor or a pharmaceutically acceptable salt, solvate or ester thereof, and said second compartment contains the pharmaceutically active agent acetylsalicylic acid or a pharmaceutically acceptable salt, solvate or ester thereof.

2. The solid pharmaceutical dosage form according to claim 1, wherein the different first and second compartments comprise first and second layers or alternatively, comprise ticagrelor containing particles and acetylsalicylic acid containing particles, respectively, further wherein one or both of said sets of particles are coated.

3. The solid pharmaceutical dosage form according to claim 2, wherein the first and second layers are separated from each other by an inert layer.

4. The pharmaceutical dosage form according to claim 2, wherein the layers or particles contain the pharmaceutically active agents within a matrix forming excipient.

5. The solid pharmaceutical dosage form according to claim 1, wherein each compartment contains at least one excipient in addition to said pharmaceutically active agent.

6. The solid pharmaceutical dosage form according to claim 1 wherein the solid oral dosage form comprises ticagrelor or a pharmaceutically acceptable salt, solvate or ester thereof, in an amount therapeutically equivalent to ticagrelor free base in an amount from 10 to 400 mg.

7. The pharmaceutical dosage form according to claim 1, wherein the solid oral dosage form comprises acetylsalicylic acid or a pharmaceutically acceptable salt, solvate or ester thereof, in an amount therapeutically equivalent to acetylsalicylic acid free acid in an amount from 5 to 500 mg.

8. The solid pharmaceutical dosage form according to claim 1, wherein said dosage form is an immediate-release dosage form or a modified-release dosage form.

9. The solid pharmaceutical dosage form according to claim 1, wherein more than 60% of ticagrelor or the pharmaceutically acceptable salt, solvate or ester thereof, is dissolved from the dosage form not later than 60 minutes as determined by USP apparatus I (Basket System) using 900 ml water containing 0.3% sodium dodecylsulfate at 37° C. as dissolution medium and a rotation speed of 75 rpm.

10. The solid pharmaceutical dosage form according to claim 1 wherein at the most 90% of ticagrelor or pharmaceutically acceptable salt, solvate or ester thereof, is dissolved from the dosage form within 10 hours as determined by USP apparatus I (Basket System) using 900 ml water containing 0.3% sodium dodecylsulfate at 37° C. as dissolution medium and a rotation speed of 75 rpm.

11. The solid pharmaceutical dosage form according to wherein more than 80% of acetylsalicylic acid or the pharmaceutically acceptable salt, solvate or ester thereof, is dissolved from the dosage form not later than 60 minutes as determined by USP apparatus I (Basket System) using 900 ml water containing 0.3% sodium dodecylsulfate at 37° C. as dissolution medium and a rotation speed of 75 rpm.

12. The solid pharmaceutical dosage form according to claim 1, wherein at the most 95% of acetylsalicylic acid or the pharmaceutically acceptable salt, solvate or ester thereof, is dissolved from the dosage form within 10 hours as determined by USP apparatus I (Basket System) using 900 ml water containing 0.3% sodium dodecylsulfate at 37° C. as dissolution medium and a rotation speed of 75 rpm.

13. A package comprising the solid pharmaceutical dosage form according to claim 1, characterized in that the package stabilizes the solid pharmaceutical dosage form.

14. The package according to claim 13, wherein the package is a water-impermeable pack that optionally comprises a desiccant.

15. A method of treating coronary artery, cerebrovascular or peripheral diseases and thrombotic events in a subject in need thereof, said method comprising the step of administering the solid dosage from according to claim 1 to said subject.

16. The solid pharmaceutical dosage form according to claim 5, wherein each compartment contains at least more than 5% active agent in its free base or acid form, based on the total weight of the compartment.

17. The solid pharmaceutical dosage form according to claim 1, wherein the solid oral dosage form comprises ticagrelor or a pharmaceutically acceptable salt, solvate or ester thereof, in an amount therapeutically equivalent to ticagrelor free base in an amount from 50 to 200 mg.

18. The solid pharmaceutical dosage form according to claim 1, wherein the solid oral dosage form comprises ticagrelor or a pharmaceutically acceptable salt, solvate or ester thereof, in an amount therapeutically equivalent to ticagrelor free base in an amount from 90 mg to 180 mg.

19. The solid pharmaceutical dosage form according to claim 1, wherein the solid oral dosage form comprises acetylsalicylic acid or a pharmaceutically acceptable salt, solvate or ester thereof, in an amount therapeutically equivalent to acetylsalicylic acid free acid in an amount from 10 to 375 mg.

20. The solid pharmaceutical dosage form according to claim 1, wherein the solid oral dosage form comprises acetylsalicylic acid or a pharmaceutically acceptable salt, solvate or ester thereof, in an amount therapeutically equivalent to acetylsalicylic acid free acid in an amount from 20 mg to 100 mg.

21. The solid pharmaceutical dosage form according to claim 1, wherein more than 75% of ticagrelor or the pharmaceutically acceptable salt, solvate or ester thereof, is dissolved from the dosage form not later than 60 minutes as determined by USP apparatus I (Basket System) using 900 ml water containing 0.3% sodium dodecylsulfate at 37° C. as dissolution medium and a rotation speed of 75 rpm.

22. The solid pharmaceutical dosage form according to claim 1 wherein at the most 80% of ticagrelor or the pharmaceutically acceptable salt, solvate or ester thereof, is dissolved from the dosage form within 10 hours as determined by USP apparatus I (Basket System) using 900 ml water containing 0.3% sodium dodecylsulfate at 37° C. as dissolution medium and a rotation speed of 75 rpm.

23. The solid pharmaceutical dosage form according to claim 1, wherein more than 95% of acetylsalicylic acid or the pharmaceutically acceptable salt, solvate or ester thereof, is dissolved from the dosage form not later than 60 minutes as determined by USP apparatus I (Basket System) using 900 ml water containing 0.3% sodium dodecylsulfate at 37° C. as dissolution medium and a rotation speed of 75 rpm.

24. The solid pharmaceutical dosage form according to claim 1, wherein at the most 90% of acetylsalicylic acid or the pharmaceutically acceptable salt, solvate or ester thereof, is dissolved from the dosage form within 10 hours as determined by USP apparatus I (Basket System) using 900 ml water containing 0.3% sodium dodecylsulfate at 37° C. as dissolution medium and a rotation speed of 75 rpm.

25. The package according to claim 13, wherein the package is a water-impermeable pack selected from the group consisting of an aluminum pack, an aluminum blister pack and a polyethylene pack, wherein the water-impermeable pack further comprises a desiccant.
